(19) United States
(12) Patent Application Publication
(10) Pub. No.: US 2018/0140651 A1
(43) Pub. Date: May 24, 2018

(54) COMPOSITION FOR PREVENTING OR TREATING LIVER DISEASE COMPRISING POGOSTEMONIS HERBA EXTRACT

(71) Applicant: RESEARCH & BUSINESS FOUNDATION SUNGKYUNKWAN UNIVERSITY, Suwon-si, Gyeonggi-do (KR)

(72) Inventors: Sun Mee LEE, Seoul (KR); Sangbin LEE, Ulsan (KR); Hongik CHO, Seoul (KR)

(73) Assignee: RESEARCH & BUSINESS FOUNDATION SUNGKYUNKWAN UNIVERSITY, Suwon-si, Gyeonggi-do (KR)

(21) Appl. No.: 15/575,949

(22) PCT Filed: Apr. 18, 2016
(86) PCT No.: PCT/KR2016/003995

§ 371 Date: Nov. 21, 2017

(30) Foreign Application Priority Data
May 22, 2015 (KR) 10-2015-0071582
Apr. 15, 2016 (KR) 10-2016-0046477

(51) Int. Cl. A61K 36/53; A61P 1/16; A61P 35/00; A23L 33/105

(52) U.S. Cl. CPC A61K 36/53; A61P 1/16; A61P 35/00; A23V 2002/00; A61K 2236/331; A61K 2236/333; A61K 2236/33; A23L 33/105

(57) ABSTRACT

The present invention relates to a composition having a Pogostemonis Herba extract which can be effectively used for preventing or treating liver disease. It has been specifically confirmed that the Pogostemonis Herba extract of the present invention can be used to effectively reduce ALT and IL-6 levels in blood, TLR 4 protein expression level within a liver. Thus, a more fundamental approach to a targeted therapy is expected to prevent or treat liver disease.

### TECHNICAL FIELD

The present invention relates to a composition for preventing or treating a liver disease, which includes a Pogostemonis Herba extract, and more specifically, a pharmaceutical composition and food composition for preventing or treating a liver disease, which includes a Pogostemonis Herba extract as an active ingredient.

### BACKGROUND ART

The liver plays various roles in metabolism of lipids in a human body, detoxification, secretion of bile, storage of various nutrients, hematopoiesis or blood coagulation, and regulation of a circulation blood volume, and is one of the essential organs for supporting life.

More specifically, in terms of the functions of the liver, first, the liver has a function of managing energy metabolism, and thus all nutrients absorbed from food are metabolized into substances capable of producing energy therein and supplies the nutrients to the entire body or stores them. Second, the liver has a function of synthesizing, storing and distributing approximately 2,000 types of enzymes, albumins, serum proteins of coagulation factors, and lipids such as bile acid, phospholipids, cholesterols, etc. Third, the liver has detoxification and degradation functions and therefore detoxifies drugs, alcohols, toxic substances, etc., and since liver cells are easily damaged, drug-induced, toxicity-induced, or alcohol-induced liver diseases frequently occur. In addition, the liver also plays an important role in secretion of various metabolites to the duodenum, an immune function, and life support.

Meanwhile, generally, the most frequent liver disease is hepatitis in which inflammation takes place in the liver, and the liver diseases may be divided into, depending on conditions, acute hepatitis and chronic hepatitis, and depending on causes, viral hepatitis, alcoholic hepatitis, drug-induced hepatitis, etc. In addition, liver diseases triggered by such disorders include fatty liver, hepatitis, liver cirrhosis, liver cancer, etc. While a mechanism involved in progression of liver diseases is not completely defined, it is known that, first, fatty liver is caused, and due to secondary cell damage, develops into progressive liver diseases such as fatty liver inflammation, liver cirrhosis, etc. In addition, since the liver disease does not have subjective symptoms at an early stage and thus is found only in its advanced stage, it ranks high as a cause of death domestically and globally.

Now, generally used methods for treating a liver disease are largely divided into a dietary regime and a drug therapy, and in most cases, both methods are used in combination. In the drug therapy for a liver disease, drugs having various action mechanisms according to the cause and type of a disease may be used, and examples of the generally used drugs include liver cell regeneration stimulants and liver function supplements such as ursodexoycholic acid, silymarine, biphenyldimethyldicarboxylate (DDB), glutathione, carnitine orotate, glycyrrhizin, and multivitamins, antiviral agents such as acyclovir, immunosuppressants such as corticosteroids, 6-mercaptopurine (6-MP), and azathioprine, and antifibrotic agents such as D-penicillamine. However, it is difficult to radically treat a liver disease due to a limited liver protective effect and side effects of these drugs.

Under this background, today, an interest in agents for treating a liver disease derived from natural substances which have excellent biocompatibility and cause less concern for side effects is growing, and such agents have become a major subject for research. However, basic research for effective amounts and exact pharmaceutical effects of most natural substances has not produced sufficient results, and therefore the natural substances are difficult to be used as an agent for treating a liver disease.

However, the pogostemon herb included in Labiatae, called “Pogostemonis Herba,” is widely cultivated in the Phillippines, India, South China, etc. The pogostemon herb refers to a dried aerial part of Pogostemon cablin Bentham, which is perennial vegetation, and is known to contain approximately 1.5% of essential oils such as pachouli alcohol, eugenol, cinnamic aldehyde, pagostol or pachouli pyridine. While the effect of treating an inflammatory intestinal disease such as ulcerative colitis or Crohn's disease with the pogostemon herb has been reported (Korean Patent Publication No. 10-2010-0136636), therapeutic effects against a liver disease have been reported to be insignificant.

### DISCLOSURE

### Technical Problem

The present invention is suggested to solve the above-mentioned problems, and the inventors confirmed the effect of decreasing levels of ALT and IL-6 in blood and the level of TLR4 protein expression in liver tissue by using a Pogostemonis Herba extract, and based on this, the present invention was completed.

Therefore, the present invention is directed to providing a pharmaceutical composition for preventing or treating a liver disease, which includes a Pogostemonis Herba extract as an active ingredient.

In addition, the present invention is directed to providing a food composition for preventing or improving a liver disease, which includes a Pogostemonis Herba extract as an active ingredient.

However, technical problems to be solved in the present invention are not limited to the above-described problems, and other problems which are not described herein will be fully understood by those of ordinary skill in the art from the following description.

### Technical Solution

To achieve the objects of the present invention, the present invention provides a pharmaceutical composition for preventing or treating a liver disease, which includes a Pogostemonis Herba extract as an active ingredient.

In an exemplary embodiment of the present invention, the Pogostemonis Herba extract may be extracted with one or more solvents selected from the group consisting of water, an alcohol having 1 to 4 carbon atoms, and a mixed solvent thereof.

In another exemplary embodiment of the present invention, the liver disease may be selected from the group consisting of hepatitis, hepatotoxicity, cholestasis, fatty liver, liver cirrhosis, hepatic ischemia, alcoholic liver disease, liver abscess, hepatic encephalopathy, liver atrophy and liver cancer.

In still another exemplary embodiment of the present invention, the composition may inhibit the increase in the level of alanine aminotransferase (ALT) and/or interleukin 6 (IL-6) concentrations in blood.

In yet another exemplary embodiment of the present invention, the composition may inhibit the increase in the level of toll-like receptor (TLR) 4 protein expression in liver tissue.

The present invention provides a food composition for preventing or improving a liver disease, which includes a Pogostemonis Herba extract as an active ingredient.

The present invention provides a health functional food composition for preventing or improving a liver disease, which includes a Pogostemonis Herba extract as an active ingredient. The present invention provides a method for treating a liver disease, which includes administering the pharmaceutical composition to a subject.

The present invention provides a use of a Pogostemonis Herba extract to produce a drug for preventing or treating a liver disease.

### Advantageous Effects

A composition according to the present invention includes a Pogostemonis Herba extract as an active ingredient, and thus it was concretely confirmed that ALT and IL-6 levels in blood and the level of a TLR4 protein expression in liver tissue using the Pogostemonis Herba extract can be effectively reduced. Therefore, the composition is expected to be effectively used as a pharmaceutical composition for preventing or treating a liver disease.

### DESCRIPTION OF DRAWINGS

FIG. 1 shows the result of comparing changes in alanine aminotransferase (ALT) level in blood according to the administration of a Pogostemonis Herba extract (25 and 50 mg/kg).

FIG. 2 shows the result of comparing changes in IL-6 level in blood according to the administration of a Pogostemonis Herba extract (25 and 50 mg/kg).

FIG. 3 shows the result of comparing changes in the level of a TLR 4 protein expression in liver tissue according to the administration of a Pogostemonis Herba extract (25 and 50 mg/kg).

### MODES OF THE INVENTION

The present invention provides a pharmaceutical composition for preventing or treating a liver disease, which includes a Pogostemonis Herba extract as an active ingredient, and the composition includes a pharmaceutical composition or a food composition.

Particularly, the present invention is extracted from a natural substance, and although administered for a long time, has insignificant side effects of a conventional therapeutic agent. The composition of the present invention has an inhibitory effect on an inflammatory response according to necrosis of liver cells and liver damage, and thus can effectively prevent or treat a liver disease.

The term “Pogostemonis Herba” used herein is included in Labiatae, is a plant widely cultivated in the Phillippines, India, South China, etc., and refers to a dried aerial part of Pogostemon cablin Bentham, which is perennial vegetation. In addition, it has been reported that the Pogostemonis Herba contains approximately 1.5% of essential oils such as pachouli alcohol, eugenol, cinnamic aldehyde, pagostol or pachouli pyridine. Meanwhile, Agastache rugosa (Korean mint) having a similar scientific name to Pogostemonis Herba is a species included in a completely different genus of origin, and the dried entire plant of Korean mint (Agastache rugosa), which is annual vegetation.

In the present invention, the Pogostemonis Herba extract may be extracted using a conventional solvent according to a conventional method known in the art to extract an extract from a natural substance, that is, under conventional temperature and pressure conditions. For example, in the present invention, the Pogostemonis Herba extract may be extracted using one or more solvents selected from the group consisting of water, an alcohol having 1 to 4 carbon atoms, and a mixed solvent thereof, and preferably, water. The water refers to water suitable for drinking standards according to the Ministry of Health, and can generally be water such as distilled water filtered using a filtering device or water purifier, sterile water, anionic water, tap water, or mineral water. The Pogostemonis Herba extract may be extracted using various methods including hot water extraction, maceration extraction, reflux extraction, and ultrasonic extraction, but the present invention is not limited thereto.

The prepared extraction is then subjected to any one or all of filtration, concentration, and drying so that a solvent may be removed. For example, the filtration may be performed using filter paper or a vacuum filter, the concentration may be performed using a vacuum concentrator, and the drying may be performed by a freeze-drying method, but the present invention is not limited thereto.

In addition, the extract extracted using the solvent may be further subjected to fractionation with a solvent selected from the group consisting of hexane, methylene chloride, acetone, ethyl acetate, ethyl ether, chloroform, water and a mixture thereof. A temperature during the fractionation may be 4° C. to 120° C., but the present invention is not limited thereto.

Meanwhile, the term “liver disease,” which is a disease to be improved, prevented or treated by the composition of the present invention, refers to impairment of one or more of various functions of the liver, and therefore, normal metabolism cannot be achieved. The most frequent type of liver disease is hepatitis, divided into acute hepatitis and chronic hepatitis, and generally, the acute hepatitis is easily treated and benign. The acute hepatitis includes virus-induced hepatitis, alcoholic hepatitis, and toxic hepatitis depending on a cause, and among these, today, virus-induced hepatitis is most frequently found. The liver diseases include one capable of being treated using a Pogostemonis Herba extract without limitation, and examples of the liver diseases may include hepatitis, hepatotoxicity, cholestasis, fatty liver, liver cirrhosis, hepatic ischemia, alcoholic liver disease, liver abscess, hepatic encephalopathy, liver atrophy and liver cancer.

The Pogostemonis Herba extract of the present invention may play a role in treating or preventing a liver disease by inhibiting the increase in level of alanine aminotransferase (AT) and/or interleukin 6 (IL-6) in blood.

The term “ALT” used herein refers to alanine aminotransferase, which is an enzyme present at a large amount in liver cells and becomes a specific marker of liver injury. A normal range of ALT is 7 to 56 units per liter of serum. The enzyme catalyzes a chemical reaction in cells by transferring an amino group from a donor molecule to a recipient molecule, and since it is present at a large amount in liver cells, is used as a specific marker indicating a liver condition.

In addition, the term “interleukin 6 (IL-6)” used herein refers to one of the inflammatory cytokines produced in adipocytes or macrophages, and used as a biological marker of an inflammatory response.

The Pogostemonis Herba extract of the present invention may play a role in treatment or prevention of a liver disease by inhibiting the increase in an expression level of a toll-like receptor (TLR) 4 protein in liver tissue.

In addition, the term “toll-like receptor (TLR) 4 protein” used herein refers to one of the receptors directly recognizing a specific molecular pattern of a component constituting a pathogen in natural immunity of early biophylaxis against pathogens such as bacteria, viruses and parasites, and used as a marker for recognizing a gram-negative bacteria-derived lipopolysaccharide (LPS).

Meanwhile, the term “prevention” used herein refers to all actions of inhibiting a liver disease or delaying the onset thereof by administration of the pharmaceutical composition according to the present invention.

In addition, the term “treatment” used herein refers to all actions involved in alleviating or beneficially changing symptoms of obesity or a lipid-related metabolic disease by administration of the pharmaceutical composition according to the present invention.

In addition, the pharmaceutical composition according to the present invention may include a pharmaceutically acceptable carrier as well as an active ingredient. Here, the pharmaceutically acceptable carrier is conventionally used in drug formulation, and includes, but is not limited to, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil. As well as the above components, the pharmaceutical composition according to the present invention may further include a lubricant, a wetting agent, a sweetening agent, a flavoring agent, an emulsifier, a suspending agent, or a preservative.

The pharmaceutical composition of the present invention may be administered orally or parenterally (e.g., intravenously, subcutaneously, intraperitoneally or locally) depending on a desired method, and a dose of the pharmaceutical composition may vary depending on the condition and body weight of a patient, the severity of a disease, a drug type, an administration route and time, and may be suitably selected by one of ordinary skill in the art.

The pharmaceutical composition of the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in medical fields. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the pharmaceutical composition of the present invention may be dependent on a patient's age, gender, condition and body weight, an absorption rate of the active ingredient in the body, an inactivation rate, an excretion rate, a type of disease, or a drug used in combination, and may be generally administered at 100 to 500 mg/kg of body weight daily or every other day, or divided into one or three daily administrations. However, the effective amount may vary depending on an administration route, the severity of obesity, sex, body weight or age, and therefore, the scope of the present invention is not limited by the dose by any means.

In one exemplary embodiment of the present invention, after hepatic ischemia was induced, a Pogostemonis Herba extract was administered to an experimental animal that had undergone reperfusion to confirm a therapeutic effect according to liver damage (refer to Example 1). As a result, in a Pogostemonis Herba extract-administered group, the decrease in ALT and IL-6 levels in blood and the level of a TLR 4 protein expression in liver tissue, which are indicators of liver injury and an inflammatory response, was identified, and it was specifically confirmed that the Pogostemonis Herba extract can be very useful as a composition for treating a liver disease (refer to Examples 2 to 4).

Here, in an exemplary embodiment of the present invention, a health functional food composition for preventing or improving a liver disease which includes a Pogostemonis Herba extract as an active ingredient may be provided.

The term “improvement” used herein refers to all types of actions that at least reduce parameters related to a condition to be treated, for example, a degree of a symptom. Here, the functional food composition may be simultaneously or separately used with a therapeutic agent before or after the onset of a corresponding disease to prevent or improve a liver disease.

The term “health functional food” used herein refers to a food group giving added value to food such that the function of corresponding food acts or is exhibited for a specific purpose using a physical, biochemical or bioengineering technique, or food processed by being designed to sufficiently express a body modulating function of a food composition for modulating a body defense rhythm or preventing and recovering from a disease in the body. The health functional food may further include a sitologically acceptable food supplementary additive, and specifically, a suitable carrier, an excipient or a diluent, which are conventionally used.

In addition, the health functional food composition of the present invention may contain various nutrients, vitamins, minerals (electrolytes), flavoring agents including synthetic and natural flavoring agents, coloring agents, fillers (cheese, chocolate, etc.), pectic acid and a salt thereof, alginic acid and a salt thereof, organic acids, protective colloidal thickening agents, pH adjustors, stabilizers, preservatives, glycerin, alcohols, or carbonizing agents used in soda, and such components may be used independently or in combination.

An amount of the Pogostemonis Herba may be contained at 0.001 to 90 wt %, and preferably 0.1 to 40 wt % of the total weight of the health functional food, and for long-term administration, the amount may be contained within the above-mentioned range, but since the active ingredient does not have a problem in terms of safety and thus can be used exceeding the above-range. Therefore, the amount is not limited to the above range.

The present invention provides a method for treating a liver disease by administering the pharmaceutical/food composition to a subject. The term “subject” refers to a target disease to be treated, and more specifically, a mammal such as a human, or a non-human primate, a mouse, a rat, a dog, a cat, a horse, and a cow.

Hereinafter, to help in understanding the present invention, exemplary embodiments will be disclosed. However, the following examples are merely provided to more easily understand the present invention, and the scope of the present invention is not limited to the examples.

### EXAMPLES

### Example 1

### Preparation and Methods for Experiment

1-1. Extraction of Pogostemonis Herba Extract

100 g of standardized Pogostemonis Herba (Oriental Medicine Land) was prepared. The Pogostemonis Herba was refluxed using deionized water as an extraction solvent at 55 to 60° C. for 6 hours and digested three times. After filtering an extract, the deionized water was removed through vacuum concentration, thereby yielding a Pogostemonis Herba extract. A yield of the final Pogostemonis Herba extract was 8%.

1-2. Experimental Animals

For experimental animals, ICR strain male mice (27 to 29 g) were provided from Daehan BioLink Co. Ltd., and acclimated for one week before being used in the experiments. All experimental animals were acclimated in a laboratory which had a regulated temperature and a 12-hour dark cycle, and solid feed (Daehan BioLink Co. Ltd.) and water were freely fed. All of the animal experiments were approved by the Animal Experiment Ethics Committee of the School of Pharmacy at Sungkyunkwan University, and carried out according to the guidelines for the National Institutes of Health (NIH publication No. 86-23, revised 1985).

1-3. Hepatic Ischemia Surgery

The mice were fasted for 18 hours, anesthetized with ketamine (6 mg/kg) and xylazine (8 mg/kg), and split along the midline at the abdomen. Hepatic ischemia was induced for sixty-minutes by clamping the hepatic artery playing a major role in oxygen supply into the left branch of a portal vein and a bile duct, and then the clamp was removed to cause reperfusion. 5 hours after the reperfusion, blood was collected from the abdominal artery, the left lobe and the median lobe of the liver were isolated and stored at −80° C. for analysis. A control group was subjected to the same process as described in the above experimental method, except that the left branch of the portal vein, the hepatic artery, and bile duct were not clamped. The Pogostemonis Herba extract was orally administered at the same time once daily from two days before surgery (48 hours and 24 hours before the hepatic ischemia surgery), and orally administered one hour before the surgery on the day of the surgery.

1-4. Statistical Analysis

All experimental results were expressed as mean±standard errors, and material analysis was conducted using an unpaired Student's t-test, and statistical significance at the P<0.05 level was determined.

### Example 2

### Confirmation of Effect of Pogostemonis Herba Extract on ALT Level in Blood during Hepatic Ischemia and Reperfusion

The change in alanine aminotransferase (ALT) activity in blood according to the administration of the Pogostemonis Herba extract was measured by a spectrophotometer (UV-1601, Simadzu, Japan) using an IVDLab kit (IBD lab, Korea).

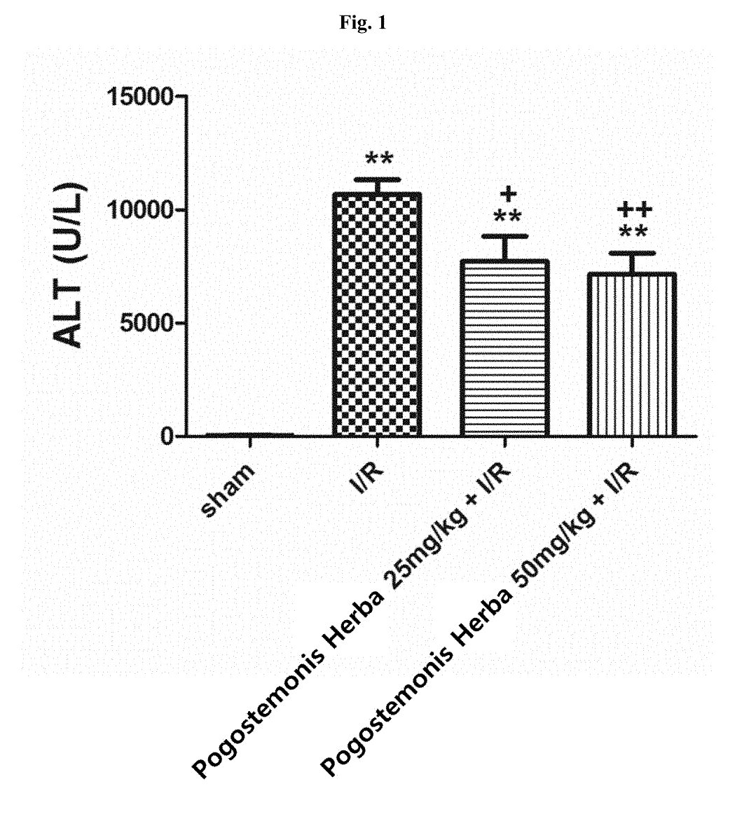

As a result, as shown in FIG. 1, ALT level in serum, which is a marker of liver injury was 44.0±12.5 U/L in the control group (sham), whereas a group (IR) subjected to reperfusion after hepatic ischemia exhibited 242.6-fold higher activity compared to the control group. However, such ALT activity was significantly inhibited in a group in which the Pogostemonis Herba extract was administered at 25 or 50 mg/kg, and a concentration-dependent tendency was shown. The result indicates that the liver injury can be effectively inhibited by administering the Pogostemonis Herba extract.

### Example 3

### Confirmation of Effect of Pogostemonis Herba Extract on IL-6 Level in Blood during Hepatic Ischemia and Reperfusion

The change in IL-6 in blood according to the treatment of the Pogostemonis Herba extract was quantified using an IL-6 ELISA kit (eBiosciences Co., San Diego, Calif., USA).

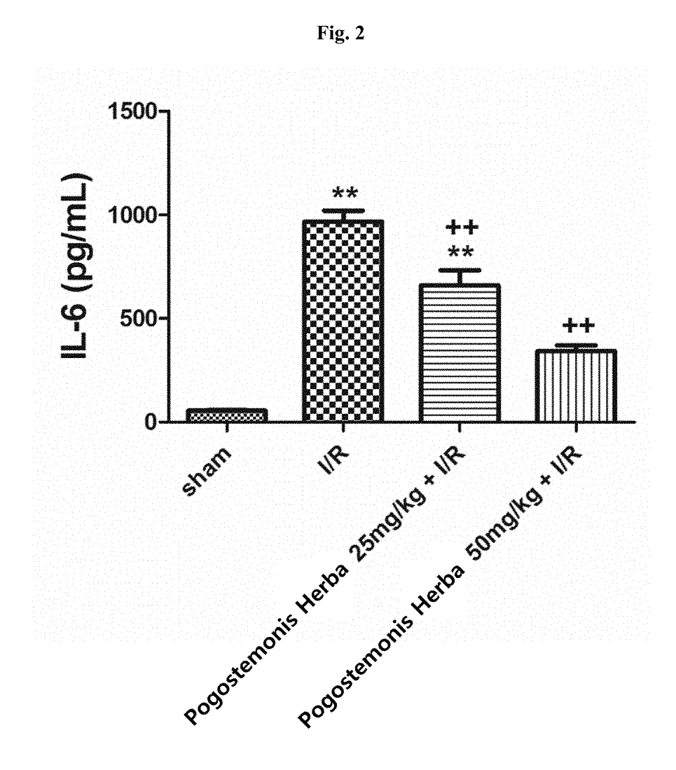

As a result, as shown in FIG. 2, an IL-6 level which is a blood inflammation marker was 55.3±2.3 pg/mL in the control group (sham), whereas a group (IR) subjected to reperfusion after hepatic ischemia exhibited a 17.5-fold higher concentration compared to the control group. However, such an IL-6 concentration was significantly inhibited in a group in which the Pogostemonis Herba extract was administered at 25 or 50 mg/kg, and a concentration-dependent tendency was shown. The result indicates that an inflammation response due to liver damage can be effectively inhibited by administering the Pogostemonis Herba extract.

### Example 4

### Confirmation of Effect of Pogostemonis Herba Extract on the Level of TLR 4 Protein Expression in Liver Tissue during Hepatic Ischemia and Reperfusion

The change in the level of TLR 4 protein expression in liver tissue according to the treatment of the Pogostemonis Herba extract was confirmed by the following method.

Proteins were extracted by homogenizing liver tissue in a 10-fold volume of a PRO-PREP™ protein extraction solution (iNtRON Biotechnology Inc., Korea), centrifuged at 10,000×g and 4° C. for 15 minutes to obtain a supernatant. The obtained supernatant was quantified using a BCA protein assay kit (Pierce Biotechnology, Ill., USA), and then heated and denatured in boiling water for 7 minutes by being mixed with 2× Western sample buffer at 1:1. The protein sample was isolated through SDS-PAGE, and then subjected to electrophoresis on a polyvinylidene fluoride membrane (Millipore, Mass., USA) using a Semi-Dry Trans-Blot Cell (Bio-Rad Laboratories, Calif., USA). The membrane having undergone electrophoresis was washed with TBS/T, and then blocked with 5% (w/v) skim milk-containing TBS/T for 1 hour at room temperature. The membrane was reacted with primary antibodies at 4° C. for 12 hours and secondary antibodies at room temperature for 1 hour, and colorized using an ECL detection system (iNtRON Biotechnology Inc.). Each band was quantified using TOTALLAB TL 120 software (Nonlinear Dynamics Ltd., Newcastle, UK). The used primary antibodies are as follows: TLR4 (1:2500 dilution, Santa Cruz Biotechnology Inc., CA, USA) and β-actin (1:5000 dilution, Sigma Aldrich, Mo., USA). Evaluation results were normalized with β-actin.

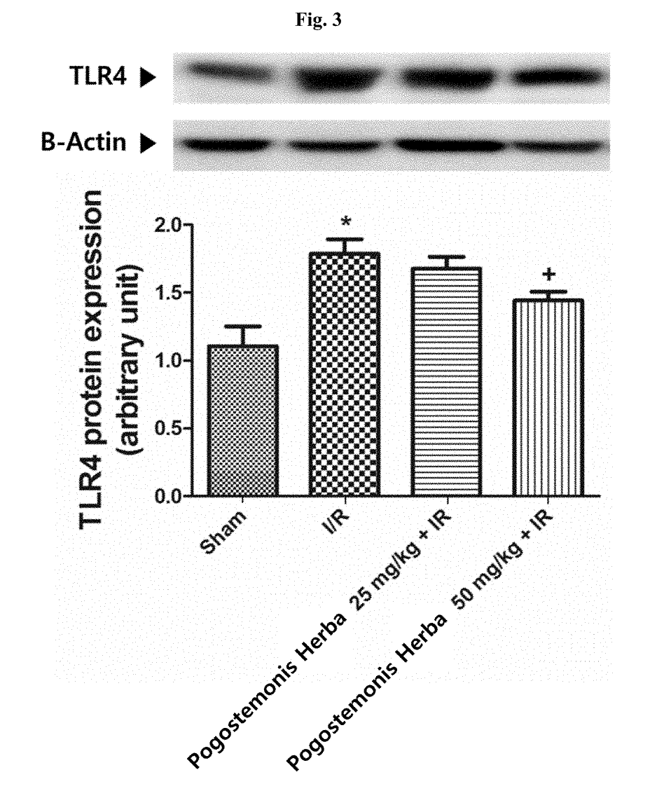

As a result, as shown in FIG. 3, after ischemia and reperfusion the level of the TLR4 protein expression in liver tissue was considerably increased 1.6 times, compared to the control group (sham). The expression level of such a TLR4 protein was significantly inhibited in the group in which the Pogostemonis Herba extract was administered at 25 or 50 mg/kg, and a concentration-dependent tendency was shown. The result indicates that an inflammation response in liver tissue can be reduced by administering the Pogostemonis Herba extract.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

It is specifically confirmed that a composition according to the present invention includes a Pogostemonis Herba extract as an active ingredient, and ALT and IL-6 levels in blood and the level of TLR4 protein expression in liver tissue are effectively reduced using the Pogostemonis Herba extract, and can be effectively used as an active ingredient of a pharmaceutical composition or food composition for preventing or treating a liver disease.

1. (canceled)

2. (canceled)

3. (canceled)

4. (canceled)

5. (canceled)

6. A food composition for preventing or improving a liver disease, comprising:
a Pogostemonis Herba extract as an active ingredient.

7. A method for treating a liver disease, comprising:
administering a pharmaceutically effective amount of pharmaceutical composition containing a Pogostemonis Herba extract as an active ingredient to a subject.

8. (canceled)

9. The method of claim 7, wherein the Pogostemonis Herba extract is extracted with one or more solvents selected from the group consisting of water, an alcohol having 1 to 4 carbon atoms, and a mixed solvent thereof.

10. The method of claim 7, wherein the liver disease is selected from the group consisting of hepatitis, hepatotoxicity, cholestasis, fatty liver, liver cirrhosis, hepatic ischemia, alcoholic liver disease, liver abscess, hepatic encephalopathy, liver atrophy and liver cancer.

11. The method of claim 7, which inhibits the increase in alanine aminotransferase (ALT) and/or interleukin 6 (IL-6) level in blood.

12. The method of claim 7, which inhibits the increase in the level of a toll-like receptor (TLR) 4 protein expression in liver tissue.
